(19) United States
(12) Patent Application Publication
(10) Pub. No.: US 2011/0144457 A1
(43) Pub. Date: Jun. 16, 2011

(54) INSTRUMENTED, COMMUNICATING PORTABLE CLOTHING AND ACCESSORY

(75) Inventor: Denis Coulon, Nimes (FR)

(73) Assignee: BODYSENS S.A.S, Nimes (FR)

(21) Appl. No.: 13/059,749

(22) PCT Filed: Aug. 4, 2009
(86) PCT No.: PCT/FR2009/000975

§ 371 Date: Feb. 18, 2011

(30) Foreign Application Priority Data
Aug. 19, 2008 (FR) 0804627

(51) Int. Cl. A61B 5/00

(52) U.S. Cl. 600/301

(57) ABSTRACT

A garment or accessory includes: at least one sensor for measuring at least one physiological parameter of the wearer of the garment or accessory; at least one element for determining the stress as a function of at least one measurement of the physiological parameter; and a remote transmission link, by wireless element, of data representative of measurements from each sensor. In some embodiments, a sensor is adapted to measure heart rate, the blood's oxygen saturation rate and the body temperature of the wearer, preferably by pinching the lobe of an ear of the wearer. For example, this sensor is an optical sensor that uses two separate wavelengths. In some embodiments, the element for determination of the composite stress determines the composite stress as function of the variability of heart rate, the body temperature, the heart rate of the user, and the voice and/or orientation.

The present invention relates to an instrumented communicating garment, including a hood or a portable accessory. It applies, in particular, to clothing and accessories, to portable sensors communicating physiological data for monitoring of, remote physiological data, the position, orientation and physical stress of the wearer and/or various hostile environment parameters. The present invention finds particular application to the protection of operational firefighters.

We know, particularly in document U.S. Pat. No. 7,298,535, a helmet equipped with sensors for capturing the body parameters of the wearer, such as the temperature inside a full-body protective slip or the temperature of external objects, with means of communication with a central station. However, this helmet does not measure the physiological parameters of the wearer and consequently does not determine his fitness, his exhaustion or physiological stress.

The present invention seeks to overcome these disadvantages.

To this end, according to a first aspect the present invention relates to a garment or a portable accessory, characterized in that it comprises:
at least one sensor for measuring at least one physiological data value of the wearer,
at least one means for determining the stress as a function of at least one measurement of a known physiological data value and
means for remote data transmission, by wireless, of the data representative of the physiological measurements from each sensor.

With these provisions, we can remotely determine whether the wearer is in good physical condition to continue its operational mission.

According to particular features, at least one sensor is adapted to measure the wearer's heart rate.

According to particular features, at least one sensor is adapted to measure blood's oxygen saturation of the wearer.

According to particular features, at least one sensor is adapted to measure the body temperature of the wearer.

According to particular features, at least one sensor adapted to measure the wearer's body temperature is an optical non-contact thermometer.

According to particular features, at least one sensor is adapted to measure heart rate, the rate of blood oxygen saturation and body temperature of the wearer. The measurements can be made in one place of the body, reducing the inconvenience to the wearer by the sensor.

According to particular features, at least one sensor clamps an ear-lobe of the wearer. With these provisions, the implementation of this sensor is simple and fast.

According to particular features, at least one sensor is an optical sensor.

According to particular features, at least one optical sensor uses at least two separate wavelengths.

With these provisions, the hood object of the present invention can measure, locally or remotely, the heart rate variability and/or blood-oxygen saturation level, for example by using photo-plethysmography.

According to particular features, at least one sensor is adapted to measure the respiration rate of the wearer.

According to particular features, at least one sensor adapted to measure the respiration rate of the wearer determines the respiration rate from oximetry data

According to particular features, at least one sensor is adapted to measure heart rate variability.

According to particular features, the hood object of the present invention includes a means for determining composite stress, according to the said variability and data representative of the body temperature and heart rate of the wearer.

According to particular features, the hood object of the present invention includes a means for determining composite stress, according to a voice signal of the wearer.

According to particular features, the hood object of the present invention includes a means for determining the composite stress, depending on the evolution of the position and/or orientation of the wearer.

According to particular features, the hood object of this invention, as succinctly described above, further comprises of at least one sensor for measuring an environmental parameter value.

According to particular features, at least one sensor for measuring an environmental parameter value includes an accelerometer.

With these provisions, it increases the reliability of cardiac, blood and/or stress-measurements during vigorous movement.

According to particular features, at least one sensor for measuring environment parameters is adapted to measure movements of the wearer.

According to particular features, at least one sensor for measuring environment parameters is suitable for measuring shock suffered to the head or the body of the wearer.

According to particular features, at least one sensor for measuring an environmental parameter value includes a noncontact thermometer adapted to measure ambient temperature.

According to particular features, at least one sensor for measuring an environmental parameter value includes a potentially toxic gas detector.

According to particular features, at least one sensor for measuring environment parameters is designed to determine the location and/or orientation of the wearer.

According to particular features, the hood object of the present invention includes a means for a point-to-point wireless audio communication with similar hoods.

With these provisions, the wearer can communicate with others.

According to particular features, the hood object of the present invention includes a means for detecting dangerous conditions for the wearer, the remote communication link being adapted to convey a signal representative of said detection.

In a second aspect, the present invention provides a method for securing a wearer of a garment or an accessory module, characterized in that it comprises:
a capture step for measuring at least the physiological parameters of the wearer of the said garment or accessory that, via sensors built in that said accessory garment or portable accessory,
a step of determining the stress as a function of at least one measurement of a physiological parameter and
a step for transmitting from a distance, by wireless, data representative of measurements from each said sensor.

In a third aspect, the present invention is a computer program, characterized in that it includes instructions executable by a processor to implement the method of the present invention, as briefly described above.

According to a fourth aspect the present invention is an information medium readable by a processor and including instructions executable by a processor to implement the method of the present invention, as briefly described above.

Benefits, goals and specific characteristics of this process, the computer program and this support information being similar to the hood object of this invention, as briefly outlined above, they are not repeated here.

Other advantages, objects and features of the present invention will become apparent from the following description made, as an explanation and in no way limiting the purpose, with the accompanying drawings, wherein:

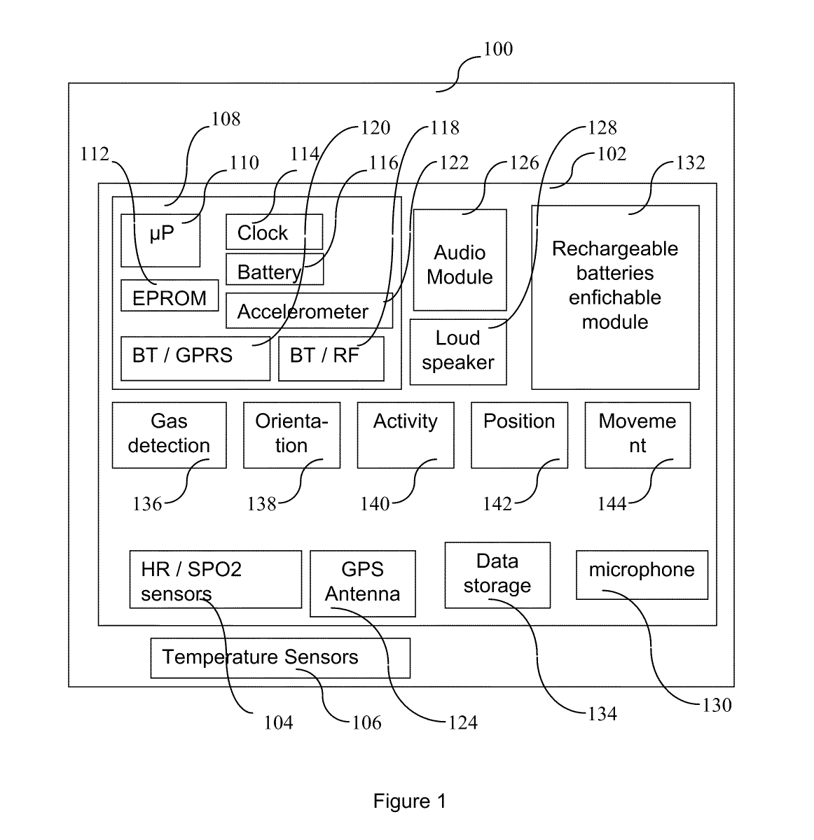

FIG. 1 shows schematically a particular embodiment of the hood object of this invention,

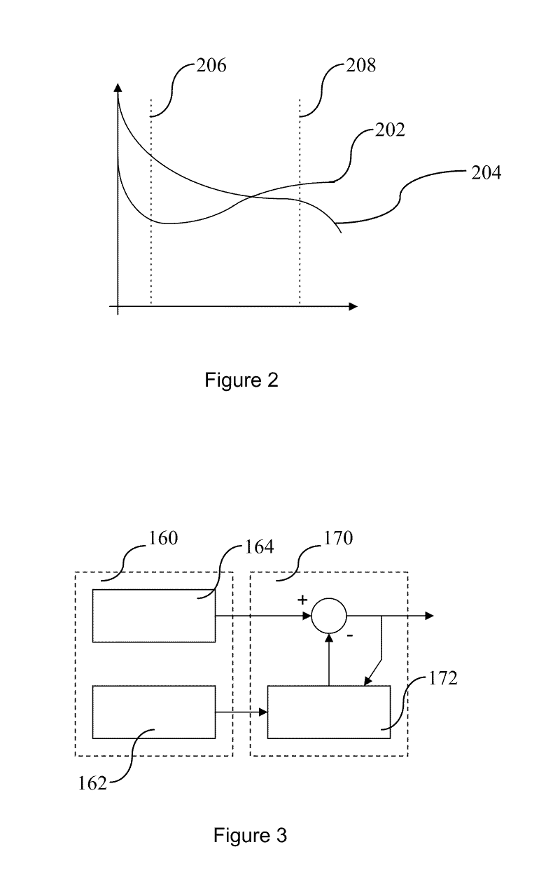

FIG. 2 shows, schematically, the optical absorption curves collected at different wavelengths for blood carrying oxygen or deoxygenated blood

FIG. 3 shows schematically an electronic circuit built into the hood shown in FIG. 1,

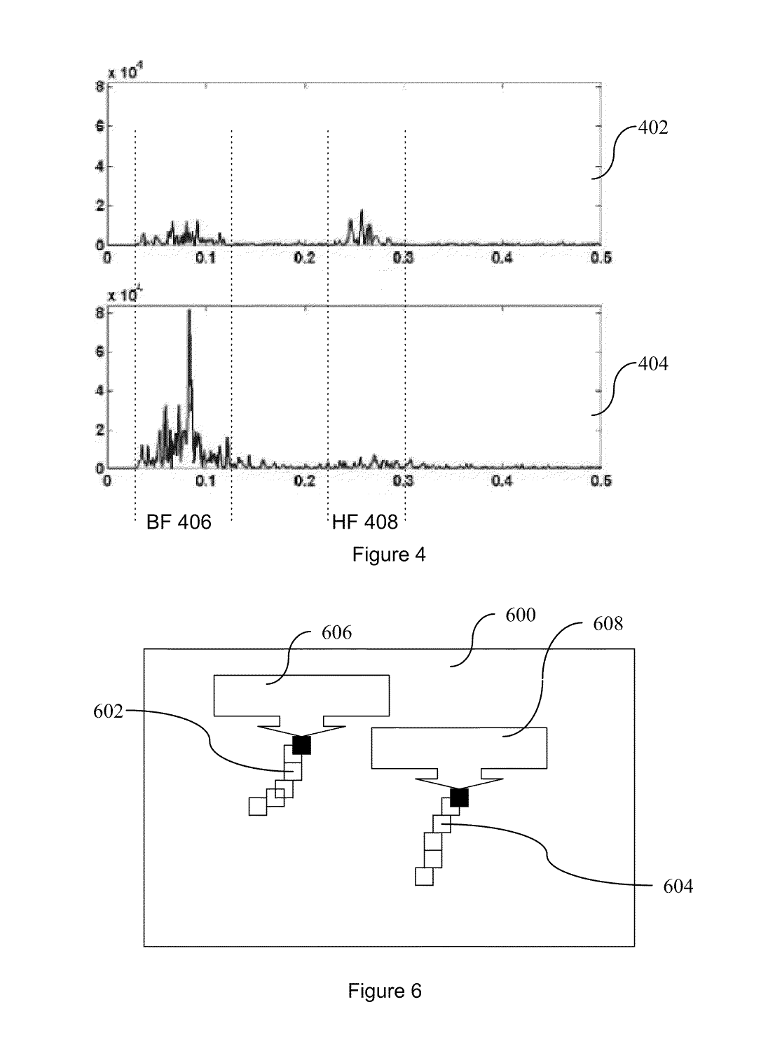

FIG. 4 shows representative frequency variability of heart rates for relaxed and stressed wearers,

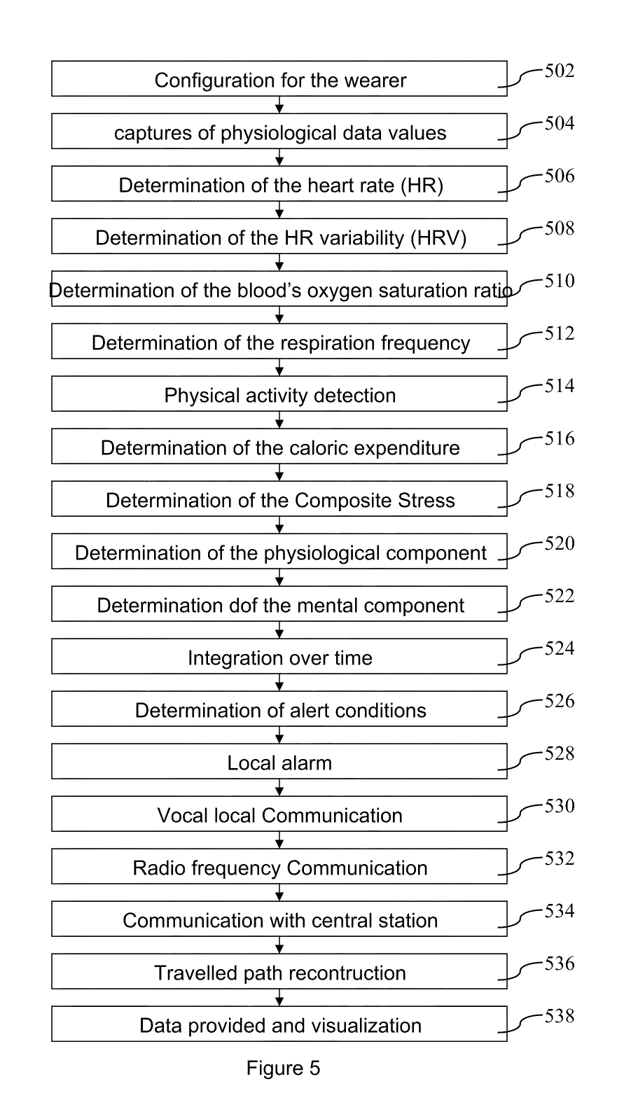

FIG. 5 shows the form of a flowchart, the steps implemented in a particular embodiment of the method of the present invention and

FIG. 6 shows, schematically, a display of geo-localized data implemented by the hood object of this invention.

Throughout the description the electronic components are shown in a hood. However, the present invention is not limited to this type of clothing but extends, rather, to any other type of clothing or accessory module, such as a jacket, an armband or helmet. In addition, the hood described can be incorporated into clothing and behaves like a hood, for example in a garment used in an industrial environment.

The purpose of the first embodiment of the hood object of this invention illustrated in FIG. 1 is to incorporate the means to detect heart rate, oxygen saturation, movement or impact, the ambient temperature and body temperature of an individual, or wearer, by positioning these means on the body of the individual. This first embodiment incorporates wireless communications capabilities, from measurements made at one place in the body. A geo-location system, e.g. GPS (which stands for Global Positioning System Global Positioning System) is integrated into the hood.

Thus, we observe in FIG. 1, a hood 100 associated with an electronic circuit 102 having electronic components implementing the following functions:
a sensor 104 for the physiological parameters of heart and blood of the wearer,
a body temperature sensor 106 of the wearer,
a Board 108 with microprocessor 110, nonvolatile memory 112, clock 114 and a battery 116,
a radio frequency communication module 118, such as Bluetooth (registered trademark) as an example.
a radio frequency communication module 120 on a mobile telephone network,
an accelerometer 122,
a module for geo-location 124,
an audio module 126,
a loudspeaker 128,
a microphone 130,
a plug-in rechargeable batteries module 132,
a module for recording and time stamping of voice 134,
a potentially toxic gas detector 136,
a module 138 for detecting orientation in space,
a module 140 for detecting the movement in space,
a standalone module 142 of detecting the position in space,
a module 144 for recovery of movement in space and
a washable dust filter (not shown) in front.

Note that some components or functions may be deported to remote sensors and, for the functions performed by a program, at distance from the wearer, a communication link enabling remote transmission of data necessary for this program.

The hood 100 is a technical garment possessing high fire resistance properties, mechanical strength, heat resistance, etc., adapted to the scope of the present invention (firemen, bikers, etc.) and offering the opportunity to remove the electronic circuits 102 for maintenance and separate the hood from these circuits 100 and 102.

The hood 100 incorporates a particle filter, made of synthetic material possibly a detachable membrane, allowing easy breathing whilst preventing dust from being inhaled by the user. The hood 100 is thus usable in hostile environments (smoke, dust, etc.) particularly including constrained environments (firefighters, etc.).

The sensor 104 for the physiological parameters for heart and blood of the wearer is, for instance, an optical “ear clip” type sensor that pinches the ear lobe, permitting the said measurements using two optical wavelengths, i.e. using two ranges of wavelengths, such as green and blue wavelengths.

Note that an ear clip style sensor for acquiring physiological information does not obstruct the ear canal and allows the normal use of conventional audio communication devices (headset phone, etc.).

The use of such an optical sensor allows the determination of heart rate, its variability and of the blood's oxygen saturation, using photo-plethysmography

The advantage of using the green light for photo-plethysmography has been described in U.S. Pat. No. 5,830,137 (Scharf), and in assessing the variability of heart rate by a method of demodulation of heart rate (see http://www.biomedical-engineering-online.com/content/01.04.1962).

It is also described in “the time-variable photoplethysmographic signal dependence of the Heart synchronous signal wavelength and sample volume,” Med. Eng. Phys. December 1995, 17 (8): 571-8 8564151. In this paper, the authors conclude that “the amplitude and signal to noise ratio (SNR) at 560 nm is greater than 940 nm for the same intensity (0.029 mWmm). A 940 nm with 1.37 and 2.77 mWmm the AC component of the signal increases with the volume of fibers while the SNR is not affected.”

The module for registration and time stamping of the voice signal 134 uses clock 114 to determine the date and time, associated with the voice signals received and transmitted by the user and then storing the associated signals.

The gas detector 136 performs detection of different gases via the integration of dedicated sensors into the hood 100. Gas levels detected are variable and can trigger alarms when values exceed predetermined limits. The gases that may be of specific concern could be carbon monoxide (CO), but also other explosive or toxic gases.

The module 138 for detecting orientation in space has at least three gyroscopes (angular rate sensors) to monitor at any moment the actual orientation in space of the wearer of the hood relative to the vertical axis and in the horizontal plane.

The detection module 140 of movement in space is capable of determining the movements in space without external reference through the integration of signals from the gyroscopes 138 and/or accelerometer 122.

The autonomous module 142 for detecting the position in space is adapted to detect the position of the wearer in space, without external reference from the information provided by gyroscopes and/or accelerometers, but with digital signal processing by temporal integration. The use of stand-alone module 142 can monitor the wearer's location in space in case of loss of GPS signal.

The module 144 of the recovery of movement in space is adapted to reconstruct the path followed by the wearer in space, from any given reference (place & time) and display this as a track in three dimensions. This module 144 permits the monitoring of the exact path of the wearer, and possibly allows the reconstruction of the path traveled, or the display of it to be transmitted to rescue teams.

The realization of the modules 138, 140, 142 and 144 can be inspired from those of navigation systems for vehicles, especially aircraft.

The washable particle filter 146, positioned in front of the hood 100, is a known type.

FIG. 2 describes the absorption levels of oxygenated blood, curve 202, and deoxygenated, curve 204, at different wavelengths, including wavelengths 206 and 208. Compared to existing systems, the reliability of measurements is augmented by the use of green or blue LEDs for a better signal to noise ratio compared to techniques used with red and infrared diodes.

The sensor 104 can thus obtain, by calculation, two primary constants “HR” (acronym for “Heart Rate”) and “SPO2” (oxygen saturation ratio).

Moreover, it is planned to use a particular combination of signal processing data derived from optical sensors and accelerometers for those placed in the hood, as shown in document IMTC 2004-Instrumentation and Measurement Technology Conference, Como, Italy, May 18-20, 2004 (see especially FIG. 6 Communication from R. Giannetti, J P Silveira, M L dotor, Golmayo D., P. Martin, F. Miguel-Tobal, A. Bilbao and Lopez-Silva S M).

FIG. 3 shows a circuit for the signal processing integrated into the hood as shown in FIG. 1, using the measurement of acceleration & movement to improve the signal to noise ratio of the measured optical signal, and improve the overall reading for processing to extract measurements of the heart rate, heart rate variability and rate of venous oximetry Given this latter information, a study of stress is possible, as discussed below. This circuit has two stages 160 and 170. We recall that in the physiological process, the source signal is distorted and perturbed as a function of body movements; the distortion signal being added to the source signal.

In the first stage, 160, a MEMS accelerometer 162 (MEMS meaning Microelectromechanical Sensor for Miniature Electromechanical Sensor) determines the acceleration of body movement, while a portable sensor 164 determines the blood signal (eg absorption at a particular wavelength). In the second stage, 170, an adaptive filter 172 receives signals from the accelerometer 162 and the portable sensor 164 and, by implementing a dynamic model, achieves active elimination of the perturbation (noise) by injecting the same signal intensity with an opposite phase.

The body temperature sensor 106 of the wearer is a non-contact optical thermometer type, eg MELEXIS MLX90614 (trademark) dual-zone measurement (infrared non-contact thermal resistance), which also captures the ambient temperature.

It is observed that the measurements of physiological parameters, via the sensors 104 and 106 are performed using a simple system positioned in one location of the body near or on the ear lobe but may, alternatively, be positioned at the wrist, temple or forehead.

Body temperature is well captured in the same place that the body measurements of PPG (acronym for “Photoplethysmography”) are made, or may be shifted to his/her head or forehead for improved accuracy. The thermometer is then fixed in the hood by a suitably positioned pocket provided in the fabric (lining) and maintained firmly against the skin.

A microprocessor 110, such as for instance, type MSP430 (trademark) has inputs and outputs as follows:

A/Entries:
analog inputs from optical sensors 104, line 1 corresponding to the first range of wavelengths,
analog inputs from optical sensors 104, line 2, corresponding to the second range of wavelengths,
analog input from temperature sensor 106,
analog input from accelerometer sensor 122,
digital switch-on or switch-off control from a switch (connected to capacitive sensors, not shown, for example type QPROX QT11 (registered trademark))
an input signal antenna (not shown),
an input from the GPS geo-location sensor 124,
analog and/or digital gas detection sensors 136,
analog and/or digital sensor to capture orientation in space 138 and
analog and/or digital motion sensors 140.

B/Outputs:
the module 120 for data transmission or mobile communication module GPRS (acronym for General Packet Radio Services), possibly after a relay implementing the Bluetooth standard,
the radio frequency communication module 118, for example implementing the Bluetooth standard,
light emitting diodes (not shown) control of switch-on or switch-off and communication,
optical sensors for power and synchronization (not shown) and
the audio module 126.

The nonvolatile memory 112 is, for example, flash EEPROM type (acronym for “Electronically Erasable Programmable Read Only Memory”) capacity of one gigabit. Battery 116 is, for example, type Li-Ion battery, for example Korea Power Cell PD3535 (trademarks).

The RF communication module 118 is a full duplex radio system between two hood wearers (pair) permitting the opportunity for external communications (a command post, etc.) and from a command post to the communicating pair by the simple action of a press button. The priority mode allows the pair to listen to the command post that interrupts the pair's communication when it broadcasts. In the example of a standard Bluetooth audio communication, the module 118 is, for example, a BT Bluegiga module (RTM) type and implements the Bluetooth standard. Alternatively, it is composed, for example by a radio frequency module CCXXXX Texas Instrument (registered trademark). The hood 100 includes radio communication and point to point contact for voice communication with other wearers of similar hoods. The hood 100 is thus fully equipped for distant reception and transmission, via wireless, of both data and digitally encoded voice, permitting open voice communication, intercom type (full-duplex), with others wearing similar equipment.

To convey audio signals to the various wearers of clothing or accessory objects of the present invention, permitting communication by radio point-to-point without manual intervention by the wearer, the hood 100 is equipped with a RF communication module 118, microphone 130 and loudspeaker 128. This module 118, microphone 130 and the loudspeaker 128 may be of conventional type or using audio bone conduction, being positioned, for example, behind the ear, near the trachea or a similarly compatible position. The communication module 118 can be integrated with the processing-communication electronics or in the form of an OEM Module (OEM stands for Original Equipment Manufacturer) as developed by Savox (brand filed). This point-to-point communication means can increase security between the closely working teammates during an actual intervention.

We note that the registration and time stamp of the voice is, alternatively, made by a remote party, for instance, the command post.

The low power radio link implemented in the module 118 can also control the activation, by means of a radio switch, of one or more light modules (not shown) of at least one external alarm (light emitting diodes, possibly flashing) set on a standalone wearer garment (vest, jacket, pants, etc.) using self adhesive tape, such as Velcro (trademark), to report the alarm status of the wearer.

The communication module 120 on a mobile phone network is, for example, combined with the geo-location module 124 type LOCOSYS including a FRACTUS (trademark) antenna for example. The two-ways communication enables to reconfigure the remote sensors for various operating modes and applications.

The accelerometer 122 is a three-axis type accelerometer, for example the accelerometer SCA 3000 (registered trademark). The incorporation of a multi-axis accelerometer helps increase the reliability of the optical measurements during intense motion (eg during sports activity) by an appropriate signal processing, detection of physical activity and caloric expenditure. For the calculation of caloric expenditure, or energy, the reader may refer to an algorithm as the OwnCal type from Polar (trademarks), based on physiological data of the wearer (size, sex, weight, age) and heart rate during exercise or exposure.

The accelerometer 122 is placed on the housing (not shown) of the optical sensor 104 or in a remote control unit, attached to the hood 100 and to the wearer's head.

The microprocessor 110 performs, in particular, a determination of the “Composite Stress”, a combination of physiological stress (some heat part) and a “mental” stress part. Thanks to accurate measurements of heart rate and high sampling rate (>1 KHz), we extract the heart rate variability. This helps assess the changing status of the health of a person, its level of physiological stress or disease development through the study of this variability (time-frequency decomposition).

Alternatively, the composite stress is measured by voice processing, for example by analysis of intonation, or by analyzing the successive orientations of the wearer in space, a change of spatial orientation more frequently repeated reflecting a higher composite stress. These different methods of determining the composite stress are alternatively implemented jointly.

The measurement of parasympathetic system via HRV (acronym for “Heart Rate Variability”) provides a simple access to the instantaneous physical and mental conditions of the individual. This means is also a factor of predictability of optimal physiological conditions.

The index of physiological stress (“Physiological Strain Index” with the acronym “PSI”) was studied in the particular case of people exposed to high temperatures to reach a broader concept including the effects of heat:

PSI=5(Tᵣₑₜ−Trₑ₀)·(39.5−Tᵣₑ₀)⁻¹+5(HRₜ−HR₀)·(180−HR₀)⁻¹

where Tᵣₑₜ and HR₂ and simultaneous measurements are taken at time t. Tᵣₑ and HR, which describe the combined load of cardiovascular and thermoregulatory system, have been associated with the same weight through the constant “5”. Thus, the index of physiological stress was spread in the range from 0 to 10, within the following values: 36.5° C.<Tre<39.5° C. and 60 ppm<HR<180 ppm, ppm here, stands for pulses per minute.

The analysis of a heart rate variability signal leads to a possibility of making an alert decision based on real conditions of the person.

For the study of stress or exhaustion we process therefore a mathematical analysis as a Fourier transform or wavelet, of the heart rate variability to separate the low frequency components (LF) and high frequency components (HF″) and one is interested in their ratio. These components represent respectively the sympathetic nervous system (LF: 0.04 to 0.15 Hz) and parasympathetic (HF: 0.15 to 0.4 Hz). For healthy people, the decline in parasympathetic activity is related to depression or tiredness, according to the article by MM. Hughes and Stoney “Psychosomatic Medicine, 62 (6): 796 (2000).

FIG. 4 illustrates the amplitudes for low frequencies LF 406 and for high frequency HF 408, in the case of the same relaxed wearer curve 402, or stressed, curve 404. As is readily understood, the ratio of average amplitudes from approximately 1 for the wearer relaxed, to greater than 3 for a stressed wearer.

The wavelet decomposition of HRV was used in conjunction with fuzzy logic by Vanderbilt University to study stress (See “Online Stress Detection using Psychophysiological Signal for Implicit Human-Robot Cooperation”, Robotica (2002) Volume 20 (6), pp. 673-686, 2002 Cambridge University Press, UK).

The “physiological” stress component is determined by calculating the PSI and the sum (overall stress) is being identified by the analysis of heart rate variability, or alternatively by the analysis of voice (intonation), or orientation in space, and after quantization, it provides an indication of the “mental” stress, the second component of the overall stress.

The Stress parameters can be presented in a form that would highlight the desired characteristics.

We define an optimal combination of these two components of physiological stress and mental stress (resting, without stressful physical environmental condition) and we add the cumulative effects (integration of the time parameter) to deduct an alert threshold, according to the individuals or their activity.

Preferably, the microprocessor 110 performs digital processing to determine the respiration rate of the wearer from oximetry data. Such a method is described in the document “A comparison of signal processing techniques for the extraction of breathing rate from the photoplethysmogram” Susannah G. Flemming and Lionel Tarassenko (Proceedings of world academy of sciences, engineering and technology, col. 24, October 2007 ISSN 1307-6884), which indicates a possibility of measuring respiration rate with an error rate of 0.04 inspirations per minute.

Applications of the hood object of this invention, including all sensors, processing electronics and communications, include the monitoring of human health in a hostile environment (fire, etc.) and that of vehicle drivers (cars, motorcycles, etc.).

The information gathered by the body sensors are transmitted by radio (Bluetooth or other Bluetooth wireless autonomous technology) to a local intermediary that may consist of an autonomous GPRS/3G module such as a mobile phone. A fully portable version is equipped with a GPRS radio or other wireless technology for data communications to a remote server. The information is sent securely to a remote server for instance using the XMPP (acronym “Extensible Messaging and Presence Protocol”).

The information transmitted to the central station, by the communication module 120 or the communication module 118, is available for viewing on a computer and accessible on the web in a format that allows a geo-location and an access to the physical and physiological parameters of each wearer of a hood object of this invention, as illustrated in FIG. 6.

We observe in FIG. 6 that, for each wearer of a hood object of the present invention (here, two wearers are represented by dark squares), markers (here, light squares) 602 and 604 represent, on a map background 600, the successive positions occupied, and a window, 606 and 608, linked to the last position occupied, provides warning data (not shown), stress, activity, heart rate, temperatures, or any other data collected or calculated as described above.

The hood object of the present invention can be spun-off to a professional version (for firefighters, for example). The electronics (see FIGS. 1 and 3) in particular can be certified to ATEX (acronym for “Atmosphere Explosive”) or other similar standards.

We observe in FIG. 5, the steps implemented in a particular embodiment of the device of the present invention, adapted to a particular embodiment of the hood object of this invention described above with reference to FIGS. 1 to 4. Although the steps illustrated in FIG. 5 are represented in a step by step mode, they are preferably implemented simultaneously by a multitasking microprocessor.

We observe in FIG. 5, that the operation of the hood begins with a step 502 of configuring the hood or the central station to associate with an identifier of the hood, the wearer's physiological data (size, sex, weight, age, and parameters of components of stress at rest).

Then, in a step 504 we capture data from at least one physiological parameter of the wearer and at least one environmental parameter of the wearer among the following:
absorption for the first range of optical wavelengths,
absorption for the second range of optical wavelengths,
blood's Oxygen saturation,
body temperature of the wearer
acceleration of the head and/or the wearer's body,
ambient temperature,
presence and rate of gas,
orientation in space,
movement in space and
geographic location of the wearer.

Then, during a step 506, we perform a determination of heart rate based on the absorption signals.

In a step 508, we perform a determination of the variability of the heart rate.

In a step 510, we perform a determination of the blood's oxygen saturation, using photo-plethysmography, according to the absorption signals.

In a step 512, we perform a determination of the respiration rate of the wearer.

In a step 514, we perform a detection of physical activity.

In a step 516, we perform a determination of caloric or energy expenditure, for example based on the wearer's physiological data (size, sex, weight, age) and heart rate during the exposure or exercise.

In a step 518, we determine the “Composite” Stress combination of physiological stress (one part from heat) and “Mental” stress by analysis of heart rate variability (time-frequency domain) based on the combined load of the cardiovascular system and the thermoregulatory system.

In a step 520, a determination of the physiological component of stress by calculating the PSI is made.

Alternatively, we determine the composite stress then the physiological stress as a function of voice processing and/or orientation in space.

In a step 522, a determination of the mental stress component from the overall stress and the physiological component of stress is performed.

In a step 524, an integration of the mental stress on a predetermined period preceding the present moment is performed.

In a step 526, a determination of alert conditions based on the integration of mental stress is performed.

In a step 528, upon detection of alert conditions, one performs a switch-on or switch-off of a warning light or local sound alarm.

In a step 530, one performs voice communication with the other local hood wearers (via the Bluetooth communication module or a radio-frequency module). During this step 530 a timestamp and a recording of voice communications is also performed.

In a step 532, one performs communication on a radio network to transmit to the central station the data collected and/or determined by the hood and the voice signals exchanged by the wearer.

In a step 534, one performs communication from the central station with the wearer through the audio module. During this step 534, optionally, one performs a reconfiguration of the sensors and/or the modes of operation, remotely via a bidirectional communication with a central station.

One notices that some determining steps described above can be remotely performed at a distance from the wearer—a remote communication link enabling the data transmission necessary for performing these programs.

In a step 536, one performs a path recovery in the space of the wearer, from a given reference (place, time), in three dimensions.

In a step 538, one provides, to the central station, the received data (data collected, determined and vocal exchanges), as an example by displaying on a computer screen and/or via an access on the web (“Web”) in a format allowing for geo-location and access to physical and physiological parameters of each wearer.

This feature lets one know the exact traveled path of the wearer of the hood and possibly permits the reconstruction of this path traveled or the visualization of it, for the purpose of sending in rescue teams.

1-27. (canceled)

28. Garment or accessory module, that comprises:
at least one sensor for measuring at least one physiological data of the wearer,
at least one means for determining the stress as a function of at least one measurement of a said physiological data and
a remote transmission link, by wireless, of data representative of measurements from each said sensor.

29. The garment or accessory module according to claim 28, wherein at least one sensor is adapted to measure the wearer's heart rate.

30. The garment or accessory module according to claim 28, wherein at least one sensor is adapted to measure the blood's oxygen saturation of the wearer

31. The garment or accessory module according to claim 28, wherein at least one sensor is adapted to measure the body temperature of the wearer.

32. The garment or accessory module according to claim 28, wherein at least one sensor clamps the lobe of an ear of the wearer.

33. The garment or accessory module according to claim 28, wherein at least one sensor is an optical sensor that uses at least two separate wavelengths.

34. The garment or accessory module according to claim 28, wherein at least one sensor is adapted to measure the respiration rate of the wearer.

35. The garment portable accessory according to claim 28, wherein at least one sensor, adapted to measure the respiration rate of the wearer determines the respiration rate from oximetry data.

36. The garment or accessory module according to claim 28, wherein at least one sensor is adapted to measure heart rate variability.

37. The garment or accessory module according to claim 36, that comprises means for determining a composite stress, according to said variability and data representative of the body temperature as well as the heart rate of the wearer.

38. The garment or accessory module according to claim 28, that comprises means of determining a composite stress, according to a voice signal of the wearer.

39. The garment or accessory module according to claim 28, that comprises means of determining a composite stress, depending on the evolution of the position and/or orientation of the wearer.

40. The garment or accessory cell according to claim 28, that further comprises at least one sensor for measuring an environment parameter value.

41. The garment or accessory module according to claim 40, wherein at least one sensor for measuring an environmental parameter value includes an accelerometer and/or is adapted to measure movements of the wearer.

42. The garment or accessory module according to claim 40, wherein at least one sensor for measuring an environmental parameter value is adapted to determine the geographical position and/or orientation of the wearer.

43. The garment or portable accessory according to claim 28, that comprises a wireless point-to-point communication means with similar garments or portable accessories.

44. The garment or accessory cell according to claim 43, that comprises means for detecting dangerous conditions for the wearer, the means for remote communication being adapted to convey a signal representing the said detection.

45. A method for securing a wearer of a garment or a portable accessory module, that comprises:
a step able to capture at least one physiological data of the wearer of the garment or accessory module that, via sensors integrated in that said accessory garment or portable accessory module.
a step for determining the stress as a function of at least one measurement of a said physiological data and
a remote transmission step, by wireless, of data representative of the measurements from each said sensor.

46. A program, that includes instructions executable by a processor for implementing the process according to claim 45.

47. An information medium readable by a processor containing instructions executable by said processor for implementing the process according to claim 45.
